(19) United States
(12) Patent Application Publication
(10) Pub. No.: US 2014/0072629 A1
(43) Pub. Date: Mar. 13, 2014

(54) BIOLOGIC MATRICES COMPRISING ANTI-INFECTIVE, METHODS AND COMPOSITIONS RELATED THERETO

(71) Applicant: MUSCULOSKELETAL TRANSPLANT FOUNDATION, Edison, NJ (US)

(72) Inventors: Carl Alexander DePaula, Cranbury, NJ (US); Arthur A. Getzman, Flemington, NJ (US); Moon Hae Suwoon, Cranbury, NJ (US)

(21) Appl. No.: 13/961,533

(22) Filed: Aug. 7, 2013

Related U.S. Application Data
(63) Continuation of application No. 12/918,991, filed on Nov. 29, 2010, now abandoned.
(60) Provisional application No. 61/030,930, filed on Feb. 22, 2008.

(51) Int. Cl. A61K 9/70; A61K 31/155; A61K 31/191; A61K 31/085

(52) U.S. Cl. CPC A61K 9/70; A61K 31/085; A61K 31/155; A61K 31/191
USPC 424/484; 514/635; 424/486; 424/488; 427/2.1

(57) ABSTRACT

Described herein are methods and compositions related to biologic matrices comprising at least one anti-infective. In certain embodiments, the invention relates to a biologic matrix comprising a slowed release anti-infective agent. In a particular embodiment, the invention relates to an acellular dermal matrix comprising a slowed release anti-infective agent, wherein the anti-infective agent is triclosan. In further embodiments, the invention relates to a biologic matrix comprising at least one anti-infective agent, wherein the biologic matrix is suitable for use in surgical procedures, such as, for example, for the replacement of damaged or inadequate integumental tissue or for the repair, reinforcement or supplemental support of soft tissue defects.

### INCORPORATION BY REFERENCE

This application claims the benefit of priority of U.S. Provisional Application No. 61/030,930, filed Feb. 22, 2008.

The foregoing application, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### BACKGROUND OF THE INVENTION

Whenever a medical device is in contact with a patient, a risk of infection is created. The risk of infection increases dramatically for invasive medical devices, such as intravenous catheters, arterial grafts, intrathecal or intracerebral shunts, and prosthetic devices, which are in intimate contact with bodily tissues and fluids.

Hernias occur when a portion of an organ or other bodily tissue protrudes through a tear in adjacent muscular tissue or fascia. Surgical reduction of the hernia can be accomplished by repositioning the herniated tissue into its original location, suturing the edges of the tear and using a reinforcing patch that is subsequently resorbed or remodeled by the body or over which scar tissue is formed. The reinforcing patch may be placed under, over, or adjacent to the tissue being repaired. Recent advancements include the use of biologically derived prosthetic material, such as patches comprising acellular human dermis, such as, for example, FlexHD® (available from the Musculoskeletal Transplant Foundation, Edison, N.J.), which is a pre-hydrated acellular dermal matrix derived from human allograft skin. These tissue-derived patches provide numerous advantages over earlier patches, such as those made from metallic or synthetic polymeric materials. Even under highly aseptic conditions, implantation requires surgical incisions in the subject, thereby increasing the likelihood of opportunistic infection. Additionally, infection may have been present in the subject prior to surgery, or a wound may have been contaminated from a traumatic incident to the patient, or improper handling of the patch prior to its use may result in microbial contamination.

Synthetic or biologic mesh used in abdominal hernia repair, for example, may become infected in patients. In an infected abdominal hernia repair, bacteria proliferate in the wound and on the implant. A serious problem with the use of a biologic mesh in infected ventral hernia applications is the tendency of the bacteria to cause the implant to be degraded and resorbed. The bacteria excrete proteolytic enzymes, which chemically react with collagen that is in the matrix and cause it to break down. When synthetic, polymeric meshes are used, the bacteria will aggregate on the mesh and proliferate, and the mesh will have to be removed in order to treat the infection.

The use of mesh in an infected site is thus problematic. It would therefore be useful to have alternative mesh, which may be useful to reduce the potential of infection during surgery or to prevent microbial infection following surgery, such as in infected ventral hernia applications.

Citation or identification of any document in this application is not an admission that such documents available as prior art to the present invention.

### SUMMARY OF THE INVENTION

Described herein are biologic matrices, which comprise at least one slowed release anti-infective agent. In some embodiments, the biologic matrix treated with at least one slowed release anti-infective agent is an acellular biologic matrix. An acellular biologic matrix as described herein can be derived from tissue selected from dermal, fascia, dura, pericardia, tendons, ligaments, and muscle. In a particular embodiment, an acellular biologic matrix of the invention is an acellular dermal matrix. In a further embodiment, the invention relates to an acellular dermal matrix comprising at least one slowed release anti-infective agent, wherein the acellular matrix is hydrated.

In some embodiments, an acellular biologic matrix of the invention is hydrated with a solution of specially denatured alcohol and water in a ratio of alcohol to water selected from the group consisting of 20:80; 25:75; 30:70; 35:65; 40:60; 45:55; 50:50; 55:45; 60:40; 65:35; 70:30; 75:25; 80:20; 85:15; 90:10; and 95:05. In a particular embodiment, the acellular biologic matrix is hydrated with a 70% specially denatured alcohol and 30% water solution. In other embodiments, the matrix is hydrated with a 40% specially denatured alcohol and 60% water solution.

In a further embodiment, an acellular biologic matrix of the invention is treated with an anti-infective agent that is triclosan. In some embodiments, the acellular biologic matrix is a hydrated acellular biologic matrix and is dipped in a triclosan solution, for example, a triclosan solution comprising about 500 to about 4000 ppm triclosan. In yet further embodiments, the dipped acellular biologic matrix comprises a concentration of triclosan of about 50 to about 2000 ppm.

In other embodiments, a hydrated acellular biologic matrix is soaked in a triclosan solution for at least about five seconds to less than about one hour, for example, a triclosan solution comprising about 500 ppm to about 4000 ppm triclosan. In other embodiments, the soaked acellular biologic matrix comprises a concentration of triclosan of about 50 to about 2000 ppm.

In further embodiments, the hydrated biologic matrix comprising anti-infective is vacuum-dried from about one minute to about six minutes.

In yet other embodiments, the hydrated acellular biologic matrix is sprayed with a triclosan solution, for example, a triclosan solution comprising about 10 ppm to about 100,000 ppm triclosan. In further embodiments, the sprayed acellular biologic matrix comprises a concentration of triclosan of about 50 to about 2000 ppm.

In certain embodiments, an acellular biologic matrix comprising anti-infective demonstrates anti-microbial activity in a zone-of-inhibition assay against gram positive bacteria. In a particular embodiment, the zone-of-inhibition assay comprises an 8 mm diameter disc of the dermal material applied to an agar plate treated with 0.5 mL of a 1:1000 dilution of Staphylococcus aureus and the zone-of-inhibition of the gram positive bacteria is at least 10 mm within about 48 hours.

In some embodiments, a treated acellular biologic matrix demonstrates toxicity against gram positive bacteria, wherein a log reduction of greater than 1.0 is observed within 24 hours.

In certain embodiments, a log reduction greater than 2.0 is observed within 24 hours.

In yet other embodiments, the invention relates to a method of treating an acellular biologic matrix with an anti-infective agent, comprising hydrating an acellular biologic matrix with a solution of about 30-70% specially denatured alcohol and dipping the acellular biologic matrix in a solution comprising an anti-infective agent, such as triclosan. In a particular embodiment, the biologic matrix is hydrated with a 40% specially denature alcohol solution.

In some embodiments, the invention relates to a method of treating an acellular biologic matrix with an anti-infective agent, comprising hydrating an acellular biologic matrix with a solution of about 30-70% specially denatured alcohol and soaking the acellular biologic matrix in a solution comprising an anti-infective agent, such as triclosan, for at least about five seconds to less than one hour. In a particular embodiment, the biologic matrix is hydrated with a 40% specially denature alcohol solution.

In some embodiments, the invention relates to a method of preparing an acellular biologic matrix for implantation into a subject, comprising hydrating an acellular biologic matrix in a solution of about 30-70% specially denatured alcohol and dipping the hydrated biologic matrix in a triclosan solution. In further embodiments, the invention relates to an acellular biologic matrix prepared by hydrating the acellular biologic matrix in a solution of about 30-70% specially denatured alcohol and dipping the hydrated biologic matrix in a triclosan solution. In a further embodiment, the acellular biologic matrix is suitable for implantation into a human patient. In a particular embodiment, the biologic matrix is hydrated with a 40% specially denature alcohol solution.

In other embodiments, the invention relates to a method of preparing an acellular biologic matrix for implantation into a subject, comprising hydrating an acellular biologic matrix in a solution of about 30-70% specially denatured alcohol and spraying the hydrated biologic matrix with a triclosan solution. In further embodiments, the invention relates to an acellular biologic matrix prepared by hydrating the acellular biologic matrix in a solution of about 30-70% specially denatured alcohol and spraying the hydrated biologic matrix with a triclosan solution. In a further embodiment, the acellular biologic matrix is suitable for implantation into a human patient. In a particular embodiment, the biologic matrix is hydrated with a 40% specially denature alcohol solution.

In some embodiments, an acellular biologic matrix treated with anti-infective is suitable for use in a surgical procedure selected from the group consisting of hernia repair, abdominal wall repair; breast reconstruction, cranial reconstruction, maxillary reconstruction, facial reconstruction, urologic reconstruction, gynecologic reconstruction, pulmonary reconstruction, bladder neck suspension, tendon repair, chronic wound care, acute wound care, burn care, dura repair and replacement, gastrointestinal reconstruction; parastomal reinforcement and repair, trauma repair, diabetic ulcer and chronic venous insufficiency ulcer.

In particular embodiments, an acellular biologic matrix treated with anti-infective is an acellular dermal matrix and is suitable for use in a surgical procedure that is a hernia repair selected from the group consisting of ventral, inguinal, paraesophageal, incisional, and hiatal.

In some embodiments, an acellular biologic matrix is treated with an anti-infective agent that is triclosan, wherein the triclosan is slowly released from the acellular biologic matrix after implantation into a human patient.

In other embodiments, an acellular biologic matrix comprises at least one additional anti-infective agent. In a particular embodiment, one anti-infective agent is triclosan and at least one additional anti-infective agent is chlorhexidine gluconate.

In yet other embodiments, an acellular biologic matrix is treated with an anti-infective agent, wherein the anti-infective agent is encapsulated in a selectively degradable polymer affixed to the acellular biologic matrix. Examples of selectively degradable polymers include polylactic glycolic acid copolymer, polycaprolactone, alginate, gelatin, collagen, carboxymethylcellulose, and hyaluronic acid.

In further embodiments, an acellular biologic matrix treated with anti-infective is lyophilized.

In some embodiments, an acellular biologic matrix comprising a slowed release anti-infective agent is derived from tissue that is derived from a mammalian source selected from the group consisting of humans, non-human primates, pigs, cows, horses, goats, sheep, dogs, cats, rabbits, guinea pigs, gerbils, hamsters, rats, or mice. In certain embodiments, the tissue source is a living human donor. In other embodiments, the tissue source is a human cadaver. In some embodiments, the source is porcine. In other embodiments, the source is equine. In yet other embodiments, the source is bovine.

In some embodiments, the invention relates to an acellular biologic matrix comprising a slowed release anti-infective agent, wherein the acellular biologic matrix has been disinfected. For example, in certain embodiments, an acellular biologic matrix, such as an acellular dermal matrix, is disinfected prior to treatment with an anti-infective agent.

In other embodiments, the invention relates to an acellular biologic matrix comprising a slowed release anti-infective agent, wherein the acellular biologic matrix has been sterilized. For example, in certain embodiments, an acellular biologic matrix, such as an acellular dermal matrix, is treated with an anti-infective agent and subsequently sterilized.

It is noted that in this disclosure and particularly in the claims, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1 is a flow chart summarizing the process of preparing a biologic matrix comprising anti-infective, as described in Example 6.

### DETAILED DESCRIPTION OF THE INVENTION

The present invention relates to a medical device comprising one or more anti-infective agents. In particular embodiments, the invention relates to a biological scaffold, such as a dermal matrix, treated with one or more anti-infective agents. Advantageously, one or more of the anti-infective agents is slowly released from the medical device over a period of time after implantation into a subject, for example, after implantation into a human patient for infected ventral hernia repair.

The biologic matrices of the invention are derived from any number of tissue sources, in particular soft tissue sources, including dermal, fascia, dura, pericardia, tendons, ligaments, and muscle.

The biologic matrices of the invention comprise one or more anti-infective agents and can be used in a wide range of surgical procedures, including general surgery and plastic or reconstructive surgery. For example, in certain embodiments, a biologic matrix comprising at least one anti-infective is suitable for use in surgical procedures for the replacement of damaged or inadequate integumental tissue or for the repair, reinforcement or supplemental support of soft tissue defects. These procedures include, but are not limited to, ventral or abdominal hernia as well as inguinal, paraesophageal, incisional, or hiatal hernia. Other surgical procedures include, but are not limited to, abdominal wall repair; breast reconstruction; cranial, maxillary, and facial reconstruction; urologic and gynecologic or pulmonary reconstructions; bladder neck suspensions; rotator cuff and other tendon repair; chronic and acute wound care; burn care; dura repair and replacement; gastrointestinal reconstructions; parastomal reinforcement and repair; trauma repairs; and diabetic ulcers and chronic venous insufficiency ulcers.

In particular embodiments, a biologic matrix of the invention is a dermal matrix. Dermal matrices suitable for use in various embodiments of the invention include biological material, such as whole tissue or tissue-derived material. In particular embodiments, a dermal matrix suitable for use in embodiments of the invention is an acellular dermal matrix.

An “acellular dermal matrix” is a tissue-derived biological matrix structure that is made from any of a wide range of collagen-containing dermal tissues by removing all, or substantially all, viable cells and all detectable subcellular components and/or debris generated by cell death. As used herein, an acellular dermal matrix lacking substantially all viable cells includes dermal matrices in which the concentration of viable cells is less than about 1% (e.g., less than 0.1%, 0.01%, 0.001%, 0.0001%, 0.00001%, or 0.000001%) of that in the tissue or organ from which the acellular dermal matrix was made. An acellular dermal matrix may also include dermal matrices comprising, after decellularization, about 25% or less of nucleic acid (e.g., DNA) that is present in normal cellularized matrix tissues.

As used herein, the term “decontamination” refers to a process or treatment that renders a medical device, instrument, or environmental surface safe to handle. For example, according to the Occupational Safety and Health Administration, decontamination is “the use of physical or chemical means to remove, inactivate, or destroy bloodborne pathogens on a surface or item to the point where they are no longer capable of transmitting infectious particles and the surface or item is rendered safe for handling, use, or disposal” [29 CFR 1910.1030].

As used herein, the term “sterile” means completely free of all living microorganisms and viruses. The terms “sterilization” or “sterilized” refer to a process, after which the probability of a microorganism surviving on a surface or item subjected to the process is less than one in one million (10⁻⁶).

The term “disinfection” refers to the elimination by physical or chemical means of nearly all pathogenic and other kinds of microorganisms but not necessarily all microbial forms. Disinfection is generally less lethal than sterilization because it destroys most recognized pathogenic microorganisms but not necessarily all microbial forms, such as bacterial spores.

As used herein, “anti-infective” refers to any compound that is capable of destroying or inhibiting the growth of a microorganism, particularly one that is pathogenic. The term “anti-infective” includes antimicrobial agents such as antibacterial and antifungal compounds.

Acellular biologic matrices can be obtained from human sources, such as, for example, dermis from elective surgery or from a cadaver, or may be obtained from non-human mammalian sources, such as non-human primates (e.g., monkeys, baboon, chimpanzees), pigs, cows, horses, goats, sheep, dogs, cats, rabbits, guinea pigs, gerbils, hamsters, rats, or mice. Generally, a biologic matrix that is treated with anti-infective and implanted into a subject will be from the same species as the intended recipient. In certain embodiments, the species will differ, e.g., in some embodiments of the invention, porcine dermis may be treated with one or more anti-infectives and transplanted into a human patient. In further embodiments, the non-human source is a genetically engineered non-human animal, e.g., one that has been genetically engineered to lack an immunogenic epitope of collagen-containing material, such as a terminal α-galactose epitope.

An acellular biologic matrix can be prepared by any suitable method known in the art. A number of suitable methods for preparing, for example, acellular dermal matrices are described in U.S. Patent Application Publication No. 20060275377 and International Application Nos. PCT/US08/52882, PCT/US08/52884, and PCT/US08/52885, which are incorporated herein by reference. Suitable methods of preparing an acellular dermal matrix from a human source include, for example, removing the epidermis from the dermal tissue, decellularizing the dermal layer below with suitable chemicals, decontaminating the tissue, and packaging the final product in a hydrated form under sterile conditions.

An example of a suitable acellular dermal matrix for use in embodiments of the invention is the human dermis from the Flex HD® product line (available from Musculoskeletal Transplant Foundation, Edison, N.J.).

In particular embodiments, the invention relates to a hydrated biologic matrix comprising at least one anti-infective. In certain embodiments, a biologic matrix of the invention is hydrated in a solution comprising 70% specially denatured alcohol and 30% water. As used herein, “specially denatured alcohol” or “SDA” refers to a solution comprising formula SDA-3C, which is listed in 21 C.F.R. part 21 subpart D 21.37; SDA-3C is 100 parts 190 proof ethanol and 5 parts isopropyl alcohol (IPA) by volume. Other suitable alcohols that may be used include isopropanol and water solutions in various ratios, e.g., 30:70; 40:60; 50:50; 70:30, 80:20, or 90:10 percent isopropanol to water. Methods of hydrating acellular biologic matrices are described in, for example, U.S. Patent Application Publication No. 20060275377 and International Application Nos. PCT/US08/52882, PCT/US08/52884, and PCT/US08/52885, which are incorporated herein by reference. Suitable methods of hydrating an acellular biologic matrix of the invention include, for example, dipping an acellular biologic matrix into a solution of 70% specially denatured alcohol and 30% water. In other embodiments, an acellular biologic matrix may be soaked for a suitable period of time, e.g., about five minutes, about ten minutes, about 15 minutes, about 30 minutes, or about one hour in a solution of 70% specially denatured alcohol and 30% water. Suitable percent ranges of alcohol that may be used include solutions comprising 10-90% SDA, e.g., 10%, 15%, 20%, 25%, 30%, 35%, 40%, 45%, 50%, 60%, 70%, 80%, or 90% SDA or other suitable alcohol.

In particular embodiments, an acellular biologic matrix treated with a 40% SDA solution is further treated with an anti-infective. A biologic matrix of the invention may be treated with an anti-infective before or after it is hydrated. For example, in some embodiments, a biologic matrix may be treated with an anti-infective agent by hydrating the matrix in a solution comprising the anti-infective. In other embodiments, a biologic matrix may be treated with an anti-infective agent by applying the anti-infective after the matrix has been hydrated, for example, first hydrating the matrix in a 40% SDA solution and then dipping the hydrated matrix in a solution comprising anti-infective.

In certain embodiments, the anti-infective is the antimicrobial agent, triclosan. The term triclosan refers to a compound also known as 2,4,4′-trichloro-2′-hydroxy-diphenyl-ether. Triclosan is an antimicrobial that is widely used in various medical applications. It is commercially available, for example, from Ciba Specialty Chemicals, Inc., Basel, Switzerland. However, its unique solubility characteristics present challenges to formulating a biologic matrix with triclosan such that the treated matrix maintains an effective amount of the anti-infective after transplantation into a subject. It is an object of the subject invention to provide a biologic matrix comprising a slowed release anti-infective. In a particular embodiment, the invention relates to an acellular dermal matrix hydrated with a 40% SDA solution and treated with an effective amount of triclosan, wherein the treated acellular dermal matrix demonstrates anti-infective activity after implantation into a subject, such as a human patient in need of treatment for an infected ventral hernia. In yet other embodiments, a biologic matrix treated with an effective amount of triclosan is suitable for prophylactic use in a subject in need thereof, e.g., to prevent infection in a patient at risk for infection.

Triclosan is very soluble in alcohol and very insoluble in water. It is an object of the subject invention, in embodiments where an acellular biologic matrix is treated with triclosan, that the triclosan demonstrates slowed release from the site of implantation of the treated matrix in a subject. In particular embodiments, an alcohol solution comprising triclosan is delivered to a hydrated acellular biologic matrix, e.g., a biologic matrix treated with a 40% SDA solution. In other embodiments, the matrix is hydrated with e.g., water, saline solution, and the like. Without being bound to theory, it is thought that the triclosan will partially separate from the alcohol phase due to the triclosan's great differential solubility (alcohol soluble and water insoluble) and deposit as an undissolved agent on the surface of and within the biologic tissue. Therefore, after implantation of the triclosan-treated biologic tissue into a subject, the triclosan will slowly exude or leach off the biologic tissue and out of the alcohol layer directly into the wound zone where bacteria may be collecting. Moreover, some of the triclosan may be held by the insoluble water phase and assist in this slow release. In this regard, where the soluble form of triclosan in alcohol would typically be active right away and dissipate within 8-12 hours in a transplant subject, such as a human abdominal hernia patient, it is thought that the precipitated triclosan in a transplanted treated acellular matrix of the subject invention will reside longer at the wound site, e.g., up to about 24-48 hours after transplantation, thereby increasing the effective amount of anti-infective available at the wound site.

In further embodiments, an acellular biologic matrix of the invention comprises an anti-infective suitable for slowed release from the biologic matrix. The term “slowed release” refers to the release of an anti-infective from the biologic matrix, wherein the anti-infective is not immediately released from the biologic matrix after implantation into a site in a subject but rather remains on the implant or leaches out over a period of time. For example, in certain embodiments, the slowed release anti-infective may be released from an implant of the invention over a period of about one hour, about two hours, about four hours, about eight hours, about ten hours, about 12 hours, about 14 hours, about 16 hours, about 18 hours, about 24 hours, about 36 hours, about 48 hours, about 72 hours, about 96 hours, about 120 hours, about two weeks, or about one month.

In certain aspects of the invention, slowed release of an anti-infective is achieved based on the solubility difference of the anti-infective in alcohol versus water, for example, where the anti-infective is highly soluble in alcohol and insoluble in water. Particularly suitable anti-infectives for use with the biologic matrices of the invention include those that are soluble in alcohol and relatively insoluble in water. In further embodiments, the anti-infective is an agent with similar solubility characteristics to triclosan. In a particular embodiment, the slowed release anti-infective is triclosan.

In other aspects, slowed release of an anti-infective is achieved by the use of one or more anti-infective agents encapsulated in selectively degradable polymers. For example, triclosan may be encapsulated in a polymer, such as poly lactic glycolic acid copolymer or polycaprolactone, and affixed to an acellular biologic matrix, for example, by dipping, soaking, printing, or spraying the encapsulated anti-infective onto the biologic matrix. Other non-limiting examples of suitable polymers for encapsulating the anti-infective agent include alginate, gelatin, collagen, carboxymethylcellulose, and hyaluronic acid. The degradable polymer comprising the anti-infective will degrade over a period of time, for example, up to about 24 hours after implantation of the treated matrix in a subject.

Methods of treating an acellular biologic matrix with anti-infective according to the invention include dipping, printing, spraying, and/or soaking the biologic matrix in a solution of anti-infective. In particular embodiments, the solution comprising anti-infective is an alcohol solution that comprises greater than 0% alcohol and is an alcohol-based solution (greater than 50% alcohol), an aqueous-based solution (greater than 50% water), or a solution comprising equal parts water and alcohol, such as a 25-70% alcohol solution comprising triclosan. In certain embodiments, the anti-infective, for example, triclosan in an alcohol solution, is applied by a spraying methodology. When spraying, the concentration of the triclosan solution applied may be controlled by passing the solution through paired squeeze rollers to remove excess fluid. An example of a spraying methodology is described in Aviv, M et al. “Gentamicin-loaded bioresorbable films for prevention of bacterial infections associated with orthopedic implants” Journal of Biomedical Materials Research Part A 83(1): 10-19 (2007), incorporated by reference herein. In yet other embodiments, the anti-infective, for example, triclosan in solution, is applied by a printing methodology. This technology is based on ink jet printing platforms and would allow printing of the triclosan in a defined pattern, if desired. An example of a printing methodology is described in Cohen, D L et al. “Direct freeform fabrication of seeded hydrogels in arbitrary geometries” Tissue Engineering 12(5): 1325-1335 (2006), incorporated by reference herein.

The term “treating” refers to coating and/or impregnating an article, such as an acellular biologic matrix, with a solution comprising an agent. Generally, the agent is an anti-infective, such as triclosan.

An acellular biologic matrix of the invention, for example, an acellular dermal matrix, may be treated with an anti-infective, such as triclosan, by quickly dipping the acellular biologic matrix in a solution of the anti-infective. In embodiments where the anti-infective is triclosan, the dermal matrix may be dipped quickly in a solution comprising about 500 to about 4000 ppm triclosan, e.g., 500 ppm, 750 ppm, 1000 ppm, 2000 ppm, 3000 ppm, or 4000 ppm.

In other embodiments, an acellular biologic matrix is treated with anti-infective by soaking the matrix in a solution comprising the anti-infective for about 5 minutes, about 10 minutes, about 15 minutes, about 30 minutes, about one hour, about two hours, or about 4 hours. In embodiments where the anti-infective is triclosan, the biologic matrix may be soaked in a solution comprising from about 100 to about 4000 ppm triclosan. In certain embodiments, the biologic matrix is soaked in a solution comprising about 300 to about 400 ppm triclosan for about 5 minutes to about 10 minutes. In other embodiments, the triclosan solution comprises about 100 to about 200 ppm triclosan and the biologic matrix is soaked for up to about one hour. In yet other embodiments, the biologic matrix is soaked in a solution comprising about 500 ppm triclosan for about 30 minutes, about 45 minutes, up to about one hour.

In yet other aspects, the biologic matrix can be treated any number of times with one or more anti-infective agents, such that the biologic matrix comprises multiple coatings or layers of one or more anti-infective agents. Accordingly, in a certain embodiment, a biologic matrix is first soaked in a solution comprising anti-infective, such as triclosan, for a period of time, and then next dipped in a solution of the same or different anti-infective. In another embodiment, a biologic matrix is soaked in a solution comprising anti-infective, such as triclosan, for a period of time, and then next soaked in a solution of the same or different anti-infective for another period of time, which may be the same or different as the first soaking time. In other embodiments, the biologic matrix is treated with a third, a fourth, or a fifth anti-infective solution.

It is to be understood that the amount of anti-infective, such as triclosan, can be titrated accordingly to achieve a suitable effective concentration that is applied to the biologic tissue. In embodiments where the anti-infective is triclosan, the amount of triclosan in solution may vary from about 100 to about 4000 ppm when applied to the biologic matrix by soaking the biologic matrix in the triclosan solution or from about 500 to about 4000 ppm when applied to the biologic matrix by dipping the biologic matrix in the triclosan solution. In yet other embodiments, the anti-infective may be applied to the biologic matrix by spraying the biologic matrix with the triclosan solution, for example an alcohol solution comprising about 100,000 ppm triclosan. Suitable triclosan concentrations for spraying a biologic matrix of the invention include from about 10 ppm to about 100,000 ppm triclosan.

It is also to be further understood that the amount of anti-infective, such as triclosan, can be titrated accordingly to achieve a suitable effective concentration that results on and/or in the biologic tissue after application of the anti-infective to the tissue. For example, in certain embodiments, the amount of anti-infective, such as triclosan, that is on and/or in the biologic tissue may vary from about 10 to about 3000 ppm, e.g., about 10 ppm, about 50 ppm, about 100 ppm, about 200 ppm, about 300 ppm, about 400 ppm, about 500 ppm, about 750 ppm, about 1000 ppm, about 1500 ppm, about 2000 ppm, about 2500 ppm, or about 3000 ppm.

In further embodiments, a biologic matrix of the invention may be lyophilized after treatment with anti-infective. For example, the matrix may be cut to a final size and then placed in a freezing and lyophilization cycle, which is thought to increase porosity of the tissue. In certain embodiments, the matrix is conditioned (e.g., equilibrated) for 15 minutes at about 20° C. or for a time period sufficient to stabilize the temperature of the matrix with a temperature ramp up from ambient with no vacuum. In further embodiments, the biologic matrix is next frozen at about −20° C. for about 80 minutes to about 120 minutes, preferably 100 minutes with no vacuum and in a further embodiment, the matrix is next frozen at about −40° C. for about 45 minutes to about 75 minutes or until the biologic matrix is frozen solid, preferably 1 hour with no vacuum.

In further embodiments, the biologic matrix is placed under vacuum, e.g., about 400 to about 800 mTorr, e.g., about 600 mTorr at −40° C. for an additional 30 minutes so that the matrix is placed under vacuum after which it is subjected to a drying phase at about −10° C. to +10° C., preferably −5° C. for about 300 to about 500 minutes, preferably 400 minutes under vacuum at, e.g., 600 mTorr. In further embodiments, the frozen lyophilized biologic matrix is then dried, e.g., at 25° C. to 35° C., preferably 25° C. for about 100 to about 400 minutes, preferably 240 minutes under vacuum at, e.g., about 400 to about 800 mTorr, e.g., 600 mTorr until the residual moisture of the lyophilized matrix is less than about 6% and the matrix has a porosity throughout its body foliated by the cavities left by the removal of the solid frozen ice. In certain embodiments, the pore cavity size left by the water being removed from the ice particles formed in the biologic matrix during the initial three freezing stages ranges from about 2.0μ to about 200μ and allows the matrix to soak up more saline or other moisturizing compound than if it was conventionally lyophilized and dried so that the same is flexible upon hydration in, e.g., an operating room.

The anti-infective activity of the treated biologic matrices of the invention can be assessed by any number of conventional means known in the art. A particularly suitable assay for assessing the anti-infective properties of a treated biologic matrix of the invention is a “zone-of-inhibition” assay. A “zone-of-inhibition assay” is an in vitro assay employing a test organism, such as methycillin-resistant staphylococcus aureus (MRSA). In a zone-of-inhibition assay, a sample of treated matrix of the invention is placed in a culture dish containing nutrients, for example, tryptic soy agar plates. The culture dish containing the treated matrix sample is then infected with a dose of the test organism, for example, a dose of MRSA. The anti-infective property of the treated biologic matrix results in a circular zone of clear area immediately adjacent to the treated biologic matrix, where the test organism is prevented from growing. Typically, the larger the circular area, the greater the anti-infective activity of the treated matrix.

Another example of a suitable assay for assessing the efficacy of an anti-infective on or in a treated biologic matrix of the invention includes inoculation and log reduction time course survivor counts. In this assay, a test organism, such as MRSA, is grown in a culture medium for approximately 24 hours and diluted to achieve a suitable concentration, such as, for example approximately 10⁵ to 10⁶ colony forming units (cfu) per square sample of treated matrix of the invention (e.g., 1″×1″). A small volume of test organism suspension is placed onto each square sample. After various time periods, the inoculated sample is placed in a Waring Blender containing media and macerated for a few seconds. Recovery is performed by plate count, for example, by plating 10 mL of a 100 mL cell culture solution containing microorganisms to give a dilution of 10⁻¹ or 1 mL to give a dilution of 10⁻². The plates are then incubated, e.g., for about 48 to 72 hours at 30 to 35° C. At the end of the incubation period, the plates are counted using a colony counter and the number of viable organisms determined. Typically, a lower amount of viable organisms compared to controls (e.g., test organism suspension without treated matrix) is indicative of anti-infective activity of the treated matrix.

A biologic matrix of the invention comprising at least one slowed release anti-infective may also comprise one or more additional anti-infectives, which may be fixed to the biologic matrix and not released from the biologic matrix after implantation into a subject. An example of an anti-infective that may be used in addition to a slowed release anti-infective of the invention is chlorhexidine gluconate.

Chlorhexidine may be provided by way of any form, salt or derivative thereof, including but not limited to chlorhexidine free base and chlorhexidine salts such as chlorhexidine diphosphanilate, chlorhexidine digluconate, chlorhexidine diacetate, chlorhexidine dihydrochloride, chlorhexidine dichloride, chlorhexidine dihydroiodide, chlorhexidine diperchlorate, chlorhexidine dinitrate, chlorhexidine sulfate, chlorhexidine sulfite, chlorhexidine thiosulfate, chlorhexidine di-acid phosphate, chlorhexidine difluoro-phosphate, chlorhexidine diformate, chlorhexidine dipropionate, chlorhexidine di-iodobutyrate, chlorhexidine di-n-valerate, chlorhexidine dicaproate, chlorhexidine malonate, chlorhexidine succinate, chlorhexidine malate, chlorhexidine tartrate, chlorhexidine dimonoglycolate, chlorhexidine monodiglycolate, chlorhexidine dilactate, chlorhexidine di-α-hydroxyisobutyrate, chlorhexidine diglucoheptonate, chlorhexidine di-isothionate, chlorhexidine dibenzoate, chlorhexidine dicinnamate, chlorhexidine dimandelate, chlorhexidine di-isophthalate, chlorhexidine di-2-hydroxynaphthoate, and chlorhexidine embonate. The teen “chlorhexidine”, as used herein, may refer to any of such forms, derivatives, or salts, unless specified otherwise. Chlorhexidine salts may be solubilized using polyethylene glycol or propylene glycol, or other solvents known in the art.

Examples of other anti-infectives that may be used to treat biologic matrices of the invention include polymyxin, bacitracin, miconazole, rifampicin, oligon, silver sulfadiazine, silver halide, silver acetate, silver iodide, ionic silver, silver zeolite, phenol, iodine, iodophor, quaternary ammonium compounds, tobramycin, gentamicin, hexetidine, polyhexamethylene biguanide, parachlorometaxylene, trichlorophenylmethyliodisalicyl, and benzalkonium chloride.

In a particular embodiment, a biologic matrix of the invention comprising at least one anti-infective is a dermal matrix that is suitable for implantation into a human patient in a medical application involving abdominal hernia repair. In certain embodiments, the abdominal hernia repair is an infected ventral hernia repair. In a further embodiment, the anti-infective is a slowed release anti-infective. In a particular embodiment, the anti-infective is triclosan.

It is thought that the addition of one or more suitable anti-infective compounds on and/or in a biological matrix of the invention, such as an acellular dermal matrix such as Flex HD®, will be effective against the bacteria and/or other flora and fauna present from a local infection or at least inhibit the growth and proliferation of these bacteria on and/or near the biological matrix. Therefore, in addition to assisting in the management of an infection per se, the anti-infective may delay the resorption or degradation of a biological scaffold, such as Flex HD®. This will allow the scaffold to function longer as a supporting, load-sharing scaffold in, for example, a repairing hernia site.

Accordingly, in certain embodiments, a biologic matrix treated with one or more anti-infective agents according to the invention is treated such that the one or more anti-infective agents prevent colonization of bacteria on the matrix and/or degradation of the biologic matrix in the presence of bacteria.

The following non-limiting examples further describe and enable one of ordinary skill in the art to make and use the present invention.

### EXAMPLES

### Example 1

### Sample Process for Dipping Acellular Dermis in a Triclosan Solution

### A. Prepare Sterile Triclosan in 70% SDA Solution:

Aseptically mix 4000 ppm Triclosan in 140 proof ethanol on the bench top in a glass bottle. Pour contents of the bottle into the top of the filtration unit and filter.

### B. Soak Dermis in Physiologic Salt Solution:

The thickness of dermis should be less than or equal to 2.5 mm.

Fill container(s) with Dulbecco's balanced salt solution (Gibco) (Invitrogen 1404141).

Any container can be used to hold the salt solution. For example, a suitable container includes a plastic Nalgene container, which holds 600 mL of solution. Another suitable container is a metal pan, which holds 1000 mL of solution. There is a typical minimum ratio of solution to cm² of dermis. The ratio is 0.69 mL salt solution/cm² dermis. Typically, there is no maximum ratio.

Place graft between two sterile wipes and squeeze out excess liquid.

Submerge the graft in Dulbecco's balanced salt solution for a minimum of 5 minutes (Note: There is typically an 8-hour maximum time for submersion).

Remove the graft from the balanced salt solution.

C. Soak Dermis in 70% SDA-3C with Triclosan:

Immediately submerge the graft in 70% ethanol solution containing 4000 ppm Triclosan for at least 5 seconds but not more than 30 seconds using a calibrated timer.

As above, any approved container can be used to hold the alcohol solution. There is a typical minimum ratio of solution to cm² of dermis. The ratio is 1.1 mL alcohol solution/cm² dermis. Typically, there is no maximum ratio.

Immediately remove tissue from solution, allow liquid on dermis to drip off for at least 5 seconds, but not more than about 60 seconds, place onto spatula, and immediately place into an impermeable foil pouch.

Seal the pouch and visually verify seal integrity.

Place the impermeable foil pouch inside a Tyvek® pouch and seal. Visually verify seal integrity.

### Example 2

### Evaluation of Dermis with Anti-Infective by Inoculation and Log-Reduction Time-Course Survivor Counts

### Method

Inoculation of the Test Material

A dilution of the 24 hours test organism to achieve a concentration of approximately 10⁵ to 10⁶ cfu per square samples approximately (1″×1″) was prepared in duplicate. 10 μL of test organism suspension was placed onto each square sample. Immediately after each time period (including an immediate recovery from product at zero-time) the inoculated sample was placed in Waring Blender containing 100 mL of GBL Stat Broth and macerated for 10-15 seconds. Recovery was performed by plate count as follows:

10 mL to give a dilution of 10⁻¹ plated with GBL Stat Agar

1 mL to give a dilution of 10⁻² plated with GBL Stat Agar

1 mL to 9.0 mL of saline and performed two fold serial dilution as 10⁻³ and 10⁻⁴ and 1.0 mL aliquots were plated with GBL Stat Agar.

The plates were incubated for 48 to 72 hours at 30 to 35° C. At the end of the incubation periods, the plates were counted using a Quebec colony counter and the number of viable organisms was determined.

Inoculation of the Control GBL Stat Broth (Numbers Control)

A dilution of the 24 hours test organism to achieve a concentration of approximately 10⁵ to 10⁶ cfu/mL was prepared. 10 μL of test organism suspension was placed in Waring Blender as with same inoculation (described above) containing 100 mL of GBL Stat Broth and macerated for 10-15 seconds. Recovery was performed by plate count as follows:

10 mL to give a dilution of 10⁻¹ plated with GBL Stat Agar

1 mL to give a dilution of 10⁻² plated with GBL Stat Agar

1 mL to 9.0 mL of saline and performed three fold serial dilution as 10⁻³ and 10⁻⁵ and 1.0 mL aliquots were plated with GBL Stat Agar.

The plates were incubated for 48 to 72 hours at 30 to 35° C. At the end of the incubation periods, the plates were counted using a Quebec colony counter and the number of viable organisms was determined.

The 0-Time controls on the product can be compared with the Numbers Control so as to determine if any immediate kill is obtained.

Groups 1-5 in the tables below refer to the following dermal matrices:

Group 1: dermis soaked in 35% ethanol with 100 ppm triclosan plus vacuum treatment.

Group 2: dermis soaked in 35% ethanol with 870 ppm triclosan plus vacuum treatment.

Group 3: dermis soaked in 35% ethanol with 18 ppm chlorhexidine gluconate plus vacuum treatment.

Group 4: dermis soaked in 35% ethanol with 28 ppm chlorhexidine gluconate plus vacuum treatment.

Group 5: dermis soaked in 0% ethanol with no anti-infective.

In Tables 1 and 2 below, the log reduction is based on Numbers Control. In Tables 3 and 4, the log reduction is based on Time 0 recovery.

TABLE 1
Test results against Staphylococcus aureus MRSA cfu survivors in duplicate A, B

| | Group 1 | | Group 2 | | Group 3 | |
|---|---|---|---|---|---|---|
| | A | B | A | B | A | B |
| Time 0 | 1.6 × 10⁶ | 1.4 × 10⁶ | 9.6 × 10⁵ | 1.2 × 10⁶ | 1.2 × 10⁶ | 1.2 × 10⁶ |
| Log₁₀ | 6.20 | 6.15 | 5.98 | 6.08 | 6.08 | 6.08 |
| 1 hour | 2.7 × 10⁵ | 3.2 × 10⁵ | 6.9 × 10⁵ | 5.5 × 10⁵ | 2.7 × 10⁵ | 2.3 × 10⁵ |
| Log₁₀ | 5.43 | 5.51 | 5.84 | 5.74 | 5.43 | 5.36 |
| Log reduction | 0.98 | 0.90 | 0.57 | 0.67 | 0.98 | 1.05 |
| 4 hours | 1.5 × 10⁵ | 2.4 × 10⁵ | 2.0 × 10⁵ | 2.4 × 10⁵ | 1.6 × 10⁵ | 2.2 × 10⁵ |
| Log₁₀ | 5.18 | 5.38 | 5.30 | 5.38 | 5.20 | 5.34 |
| Log reduction | 1.23 | 1.03 | 1.11 | 1.03 | 1.21 | 1.07 |
| 24 hours | 5.3 × 10⁴ | 6.3 × 10⁴ | 70 | 50 | 4.1 × 10⁴ | 4.1 × 10⁴ |
| Log₁₀ | 4.72 | 4.80 | 1.85 | 1.70 | 4.61 | 4.61 |
| Log reduction | 1.48 | 1.35 | 4.13 | 4.38 | 1.47 | 1.47 |

TABLE 2
Test results against Staphylococcus aureus MRSA cfu survivors in
duplicate A, B

| | Group 4 | | Group 5 | |
|---|---|---|---|---|
| | A | B | A | B |
| Time 0 | 1.6 × 10⁶ | 1.4 × 10⁶ | 1.4 × 10⁶ | 1.5 × 10⁶ |
| Log₁₀ | 6.20 | 6.18 | 6.15 | 6.18 |
| 1 hour | 2.6 × 10⁵ | 1.3 × 10⁵ | 1.1 × 10⁵ | 1.4 × 10⁶ |
| Log₁₀ | 5.41 | 5.11 | 6.04 | 6.15 |
| Log reduction | 1.00 | 1.30 | 0.37 | 0.26 |
| 4 hours | 1.6 × 10⁵ | 2.1 × 10⁵ | 1.6 × 10⁶ | 1.8 × 10⁶ |
| Log₁₀ | 5.20 | 5.32 | 6.20 | 6.26 |
| Log reduction | 1.21 | 1.09 | 0.21 | 0.15 |
| 24 hours | 5.1 × 10⁴ | 3.2 × 10⁴ | 1.9 × 10⁶ | 1.7 × 10⁶ |
| Log₁₀ | 4.71 | 4.51 | 6.28 | 6.23 |
| Log reduction | 1.49 | 1.67 | None | None |

TABLE 3
Test results against Staphylococcus aureus MRSA cfu survivors in duplicate A, B

| | Group I | | Group 2 | | Group 3 | |
|---|---|---|---|---|---|---|
| | A | B | A | B | A | B |
| Time 0 | 1.6 × 10⁶ | 1.4 × 10⁶ | 9.6 × 10⁵ | 1.2 × 10⁶ | 1.2 × 10⁶ | 1.2 × 10⁶ |
| Log₁₀ | 6.20 | 6.15 | 5.98 | 6.08 | 6.08 | 6.08 |
| 1 hour | 2.7 × 10⁵ | 3.2 × 10⁵ | 6.9 × 10⁵ | 5.5 × 10⁵ | 2.7 × 10⁵ | 2.3 × 10⁵ |
| Log₁₀ | 5.43 | 5.51 | 5.84 | 5.74 | 5.43 | 536 |
| Log reduction | 0.77 | 0.64 | 0.14 | 0.34 | 0.65 | 0.72 |
| 4 hours | 1.5 × 10⁵ | 2.4 × 10⁵ | 2.0 × 10⁵ | 2.4 × 10⁵ | 1.6 × 10⁵ | 2.2 × 10⁵ |
| Log₁₀ | 5.18 | 5.38 | 5.30 | 5.38 | 5.20 | 5.34 |
| Log reduction | 1.02 | 0.77 | 0.68 | 0.70 | 0.88 | 0.74 |
| 24 hours | 5.3 × 10⁴ | 6.3 × 10⁴ | 70 | 50 | 4.1 × 10⁴ | 4.1 × 10⁴ |
| Log₁₀ | 4.72 | 4.80 | 1.85 | 1.70 | 4.61 | 4.61 |
| Log reduction | 1.48 | 1.35 | 4.13 | 4.38 | 1.47 | 1.47 |

TABLE 4
Test results against Staphylococcus aureus MRSA cfu survivors in
duplicate A, B

| | Group 4 | | Group 5 | |
|---|---|---|---|---|
| | A | B | A | B |
| Time 0 | 1.6 × 10⁰ | 1.4 × 10⁶ | 1.4 × 10⁶ | 1.5 × 10⁶ |
| Log₁₀ | 6.20 | 6.18 | 6.15 | 6.18 |
| 1 hour | 2.6 × 10⁵ | 1.3 × 10⁵ | 1.1 × 10⁶ | 1.4 × 10⁶ |
| Log₁₀ | 5.41 | 5.11 | 6.04 | 6.15 |
| Log reduction | 0.79 | 1.07 | 0.11 | 0.03 |
| 4 hours | 1.6 × 10⁵ | 2.1 × 10⁵ | 1.6 × 10⁶ | 1.8 × 10⁶ |
| Log₁₀ | 5.20 | 5.32 | 6.20 | 6.26 |
| Log reduction | 1.00 | 0.86 | None | None |
| 24 hours | 5.1 × 10⁴ | 3.2 × 10⁴ | 1.9 × 10⁶ | 1.7 × 10⁶ |
| Log₁₀ | 4.71 | 4.51 | 6.28 | 6.23 |
| Log reduction | 1.49 | 1.67 | None | None |

### Example 3

### Evaluation of Dermis with Anti-Infective by Zone-of-Inhibition Assay

### Inoculum Preparation

The Staphylococcus aureus (MRSA) was grown into 15 mL of Trypticase Soy Broth at 30 to 35 C for 18 to 24 hours and then diluted 1:1000 in sterile saline.

### Procedure

The recovery media plates were surface-streaked with 0.5 mL of a 1:1000 dilution of Staphylococcus aureus (MRSA) (in duplicate), by sterile swab horizontally and vertically. 8 mm diameter discs (test materials) were placed approximately in the center on the surface of the agar. The plates were incubated aerobically for 48 hours at 30 to 35° C. and the zone around the test material was measured and reported (diameter in mm) at 24 and 48 hours incubation period.

### Results

TABLE 5
Zone of Inhibition Test Result after 24 and 48 hours incubation

| | Staphylococcus aureus (MRSA) | |
|---|---|---|
| Test Material | 24 hours | 48 hours |
| Acellular dermis | 25 mm/26 mm | 25 mm/26 mm |

### Example 4

### Examples of Dermis Treated with Triclosan

A.

A solution containing 100,000 ppm Triclosan in a solvent of 200 proof ethanol was made. A 1″ square piece of dermis was sprayed, using an airbrush, on each side for 5 seconds.

B.

Samples of dermis were dipped in a solution of 70% specially denatured alcohol at varying levels of Triclosan for 5 seconds and tested for zone of inhibition (ZOI). The ratio of solution volume to square cm of dermis was controlled. The levels of Triclosan tested were 2000, 4000, 6000, and 8000 ppm. All samples exhibited a zone.

C.

Samples of dermis were dipped in a solution of 70% specially denatured alcohol at varying levels of Triclosan for 30 seconds. The ratio of solution volume to square cm of dermis was controlled. The levels of Triclosan tested were 2000, 3000, and 4000 ppm. The same samples of dermis were dipped in a solution of 70% specially denatured alcohol at varying levels of Triclosan for 5 seconds and tested for ZOI. The ratio of solution volume to sq. cm of dermis was controlled. The levels of Triclosan tested were 2000, 3000, and 4000 ppm. All samples exhibited a zone.

D.

Samples of dermis were soaked in a solution of 70% specially denatured alcohol at varying levels of Triclosan for either 5 minutes or 5 hours and tested for ZOI. The ratio of solution volume to square cm of dermis was controlled. The levels of Triclosan tested were 500, 1000, and 2000 ppm. All samples exhibited a zone.

E.

In other embodiments, Triclosan dermis is soaked in 25-35% SDA long enough to equilibrate the dermis with the alcohol. The Triclosan in the alcohol solution is at or near its saturation point. For example, for 25% alcohol, the saturation point for Triclosan is 600 ppm, and for 30% alcohol, the saturation point is 1700 ppm. After the dermis has been soaked, the alcohol (ethanol) can be removed from the dermis, causing the Triclosan to precipitate inside the dermis. The alcohol can be removed by several means, such as using a vacuum chamber (low pressure), airflow over and around the dermis (evaporation), or by dipping or soaking the dermis in an aqueous solution, thus diluting the alcohol. The dermis will then contain Triclosan precipitate inside of it. It is believed that the precipitated Triclosan will be slowly released from the dermis over time after it has been implanted into the patient, due to the low solubility of Triclosan in aqueous solutions. The slow release of the Triclosan out of the dermis will inhibit the growth of microorganisms in and around the dermis, thus preserving the matrix when implanted in an infected site, and allow the soft tissue defect to heal.

### Example 5

### Examples of Methods of Preparing Dermis with Anti-Infective

A. Method of Preparing Dermis (Flex HD®) with Anti-Infective: 35% SDA with Triclosan
1. Process dermis and cut into a specific size, for example, 4 cm wide×7 cm long.
2. Prepare anti-microbial (AM) solution:
   a. Weigh 100 g 70% SDA 3C (density=0.887 g/cc)
   b. Weigh 78.8 mg Triclosan (IRGACARE MP) (Triclosan dose is 0.300 mg/g of Flex)
   c. Add Triclosan to 70% SDA, stir
   d. Weigh 100 g H₂O
   e. Add H₂O to 70% SDA with Triclosan (Item c), stir
   f. Sterile filter before use
3. Soak dermis (Flex HD) in 35% SDA for 60 minutes at room temperature with orbital shaker set to 75 rpm.
4. Drip dry, blot bottom corner
5. Place dermis into Tyvek® and seal Tyvek®
6. Place dermis inside sealed Tyvek® into vacuum chamber at room temperature for 6 minutes.
7. Remove from chamber, seal in impermeable foil pouch.
8. FlexHD with AI is ready
9. Do not rinse/soak before implanting.
   B. Method to Prepare Dermis (Flex HD®) with Anti-Infective: 35% SDA with Chlorhexidine Gluconate and Triclosan
1. Process dermis into 4×7 cm
2. Prepare anti-infective (AI) solution:
   a. Weigh 100 g 70% SDA 3C (density=0.887 g/cc)
   b. Weigh 78.8 mg Triclosan (IRGACARE MP) (Triclosan dose is 0.300 mg/g of Flex)
   c. Add Triclosan to 70% SDA, stir
   d. Weigh 100 g H₂O
   e. Add H₂O to 70% SDA with Triclosan (Item c), stir
   f. Measure 63.8 uL chlorhexidine gluconate (20% solution) (Chlorhexidine dose is 0.02715 mg/g of Flex)
   g. Add to 35% SDA, stir AI solution
   h. Sterile filter before use
3. Soak dermis (Flex HD) in 35% SDA for 60 minutes at room temperature with orbital shaker set to 75 rpm.
4. Drip dry, blot bottom corner
5. Place dermis into Tyvek® and seal Tyvek®
6. Place dermis inside sealed Tyvek® into vacuum chamber at room temperature for 6 minutes.
7. Remove from chamber, seal in foil.
8. FlexHD with AI is ready
9. Do not rinse/soak before implanting.
   C. Method to Prepare Dermis (Flex HD®) with Anti-Infective: 70% SDA with Chlorhexidine Gluconate
1. Process dermis into 4×7 cm
2. Prepare anti-infective (AI) solution:
   a. Weigh 100 g 70% SDA 3C (density=0.887 g/cc)
   b. Measure 63.8 uL chlorhexidine gluconate (20% solution) (Chlorhexidine dose is 0.02715 mg/g of Flex)
   c. Add to 70% SDA, stir AI solution
   d. Sterile filter before use
3. Soak dermis (Flex HD) in 70% SDA for 60 minutes at room temperature with orbital shaker set to 75 rpm.
4. Drip dry, blot bottom corner
5. Place dermis into Tyvek® and seal Tyvek®
6. Place dermis inside sealed Tyvek® into vacuum chamber at room temperature for 6 minutes.
7. Remove from chamber, seal in foil.
8. FlexHD with AI is ready
9. Do not rinse/soak before implanting.
   D. Method to Prepare Dermis (Flex HD®) with Anti-Infective: 70% SDA with Triclosan
1. Process dermis into 4×7 cm
2. Prepare anti-infective (AI) solution:
   a. Weigh 100 g 70% SDA 3C (density=0.887 g/cc)
   b. Weigh 78.8 mg Triclosan (IRGACARE MP) (Triclosan dose is 0.300 mg/g of Flex)
   c. Add Triclosan to 70% SDA, stir
   d. Sterile filter before use
3. Soak dermis (Flex HD) in 70% SDA for 60 minutes at room temperature with orbital shaker set to 75 rpm.
4. Drip dry, blot bottom corner
5. Place dermis into Tyvek® and seal Tyvek®.
6. Place dermis inside sealed Tyvek® into vacuum chamber at room temperature for 6 minutes.
7. Remove from chamber, seal in foil.
8. FlexHD with AI is ready
9. Do not rinse/soak before implanting.
   E. Method to Prepare Dermis (Flex HD®) with Anti-Infective: 70% SDA with Chlorhexidine Gluconate and Triclosan
1. Process dermis into 4×7 cm
2. Prepare anti-infective (AI) solution:
   a. Weigh 100 g 70% SDA 3C (density=0.887 g/cc)
   b. Weigh 78.8 mg Triclosan (IRGACARE MP) (Triclosan dose is 0.300 mg/g of Flex)
   c. Add Triclosan to 70% SDA, stir
   d. Measure 63.8 uL chlorhexidine gluconate (20% solution) (Chlorhexidine dose is 0.02715 mg/g of Flex)
   e. Add to 70% SDA, stir AI solution
   f. Sterile filter before use
3. Soak dermis (Flex HD) in 70% SDA for 60 minutes at room temperature with orbital shaker set to 75 rpm.
4. Drip dry, blot bottom corner
5. Place dermis into Tyvek® and seal Tyvek®
6. Place dermis inside sealed Tyvek® into vacuum chamber at room temperature for 6 minutes.
7. Remove from chamber, seal in foil.
8. FlexHD with AI is ready
9. Do not rinse/soak before implanting.
   F. Method to Prepare Dermis (Flex HD®) with Anti-Infective: 35% SDA with Chlorhexidine Gluconate
1. Process dermis into 4×7 cm
2. Prepare anti-infective (AI) solution:
   a. Weigh 100 g 70% SDA 3C (density=0.887 g/cc)
   b. Weigh 100 g H₂O
   c. Add H₂O to 70% SDA, stir
   d. Measure 63.8 uL chlorhexidine gluconate (20% solution) (Chlorhexidine dose is 0.02715 mg/g of Flex)
   e. Add to 35% SDA, stir AI solution
   f. Sterile filter before use
3. Soak dermis (Flex HD) in 35% SDA for 60 minutes at room temperature with orbital shaker set to 75 rpm.
4. Drip dry, blot bottom corner
5. Place dermis into Tyvek® and seal Tyvek®
6. Place dermis inside sealed Tyvek® into vacuum chamber at room temperature for 6 minutes.
7. Remove from chamber, seal in foil.
8. FlexHD with AI is ready
9. Do not rinse/soak before implanting.

### Example 6

### Process for Soaking Decellularized Dermis in a Triclosan Solution and Adjusting the Amount of Residual Alcohol in the Dermis

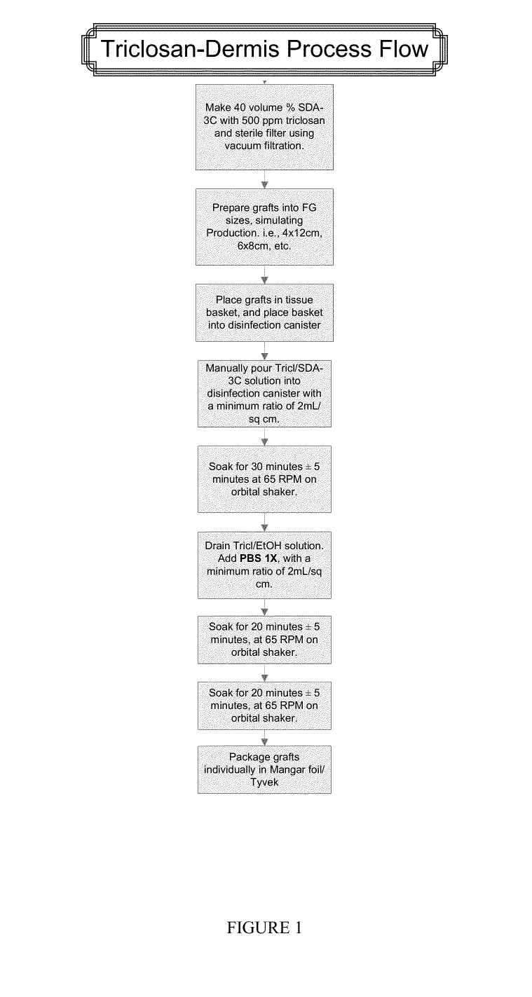

The following is procedure for preparing a biologic matrix comprising anti-infective. This procedure is also summarized in FIG. 1

### A. Prepare 40 V % SDA-3C Alcohol Solution on Bench Top:

To make a batch size of 1050 mL of 40% alcohol from 70% SDA-3C alcohol, by volume:
1. Measure out 600 mL of 70% SDA-3C alcohol. Add to glass bottle.
2. Measure out 450 mL of DI water. Add 450 mL of DI water to the 70% SDA-3C alcohol in glass bottle.
3. Mix well by placing cap on glass bottle and manually shaking.
4. Record 70% SDA-3C lot number and liquid volumes.
   Larger batch sizes can be made using the same ratio of 70% SDA-3C alcohol and DI water in order to create the desired amount of 40% SDA-3C alcohol by volume.

### B. Make 500 ppm Triclosan in 40 V % SDA-3C on Bench Top:

1. Weigh out 900 grams of 40 V % SDA-3C in glass bottle.
2. Weigh 0.45 grams triclosan.
3. Add 0.45 grams triclosan to the 40 V % SDA-3C by tipping weigh pan so that contents pour into the glass bottle; using a disposable 3 mL pipette to remove some of the solution from the glass bottle and squirt into weigh pan (the ethanol in the solution should dissolve residual triclosan); and tipping weigh pan so that the solution with residual triclosan pours into the glass bottle.
4. Mix well by placing cap on glass bottle and manually shaking until triclosan is no longer visible in the solution.
5. Enter Bio-hood, sterile filter 40 V % SDA-3C with 500 ppm Triclosan.
6. Pass supplies for sterile filtration into the Bio-hood using aseptic technique
7. Pass in tubing, the Nalgene sterile filtration unit, and sterile wipes.
8. Attach one end of the tubing to the Nalgene sterile filtration unit, and pass the other end of the tubing out of the hood.
9. Attach the other end of the tubing to the vacuum pump.
10. Wrap outside of the bottles with sterile wipes wetted with Sporklenz and pass into Bio-hood using aseptic technique.
11. Handle bottles in the hood only with sterile wipes. No other material should touch to the bottle. Change gloves after handling.
12. Sterile filter the solutions with a Nalgene 1000 mL vacuum filtration units with PES membrane.
13. Pour solution into the top of the vacuum filtration unit.
14. Replace cap on top of the container to ensure minimal evaporation of solution.
15. Turn on the vacuum pump and allow it to run until all of the solution has passed through the vacuum filtration unit.
16. Protect solution from light by covering bottle with sterile wipe.
17. Soak Dermis in SDA-3C Alcohol/Triclosan:
18. Pass all remaining sterile supplies into Bio-hood using aseptic technique.
19. Pass in the Sterile ruler, scalpel, wipes, Neoprene tubing, Canister Base, Canister Lid, Mesh Basket Assembly, and Saline.
20. Measure thickness of dermis using the thickness gauge to ensure it is between 0.4 mm-2.5 mm thick.
21. Cut the tissue to the appropriate size using a disposable scalpel and sterile wipes. Calculate total area of tissue, and the minimum liquid volume to be used.
22. Place the tissue inside the wire mesh basket and place inside the disinfection canister, which is set upon the orbital shaker.
23. Fill the canister with 40 V % SDA-3C with 500 ppm triclosan, with a minimum ratio of 2 mL solution per 1 square cm of tissue.
24. Place the lid on the canister to guard against splashing liquid.
25. Turn the orbital shaker on to 65 rpm for 30 minutes±5 minutes.
26. After the SDA-3C solution soak, turn off orbital shaker and pump the solution out of the canister using the Masterflex peristaltic pump and dispose of liquid.

### C. Setting Up the Masterflex Peristaltic Pump

1. Submerge one end of black Neoprene tubing into the bottom of the 4 L Canister and pass the other end of the tubing out of the hood. The pump drive and digital modular drive are outside of the hood.
2. Load the tubing into the peristaltic pump
3. Be sure the pump is off.
4. Open the pump.
5. Load the ⅜″ Masterflex Norprene tubing into the pump. Ensure the tubing is not pinched, or else the flow will be obstructed. Leave enough extra tubing on the end so that it reaches inside the waste collection container.
6. Close the pump.
7. Set the Occlusion Adjustment on the top of the pump head to 2.5
8. Turn pump on and allow pump to run until no more liquid is left in the canister.

9. Turn off pump when finished.

10. Dispose of the Ethanol solution in the hazardous waste container.

### D. First Alcohol Adjustment Soak

1. Fill the canister with PBS 1× w/o calcium and magnesium, with a minimum ratio of 2 mL sodium chloride per 1 square cm of tissue.
2. Place the lid on the canister to guard against splashing liquid.
3. Turn the orbital shaker on to 65 rpm for 20 minutes±5 minutes.
4. After the first PBS soak, turn off the orbital shaker and pump the saline out of the canister using the Masterflex peristaltic pump as per step 5.4.9. Turn off pump when done. PBS can be discarded in chemical sink.

### E. Second Alcohol Adjustment Soak

1. Fill the canister with PBS 1× w/o calcium and magnesium, with a minimum ratio of 2 mL sodium chloride per 1 square cm of tissue.
2. Place the lid on the canister to guard against splashing liquid.
3. Turn the orbital shaker on to 65 rpm for 20 minutes±5 minutes.
4. After the first PBS soak, turn off the orbital shaker and pump the saline out of the canister using the Masterflex peristaltic pump as per step 5.4.9. Turn off pump when done. PBS can be discarded in chemical sink.

### F. Packaging & Labeling

1. Place tissue on spatula and transfer tissue into Mangar pouch.
2. Double seal each pouch using Impulse sealer with timer set to 7.

### G. Check Seal for Integrity as Follows:

1. Observe seal from a distance of 30 to 45 cm.
2. Inspect sealed area for completeness and uniformity.
3. Identify any part of the seal where channels appear across the entire seal width.
4. If channels appear, repackage the tissue.
5. Label with sample description, reference number, and date.
6. Place Sealed Mangar pouch into Tyvek pouch. Double seal each pouch using Impulse sealer with timer set to 3.
7. Check seal for integrity as follows:
8. Observe seal from a distance of 30 to 45 cm.
9. Inspect sealed area for completeness and uniformity.
10. Identify any part of the seal where channels appear across the entire seal width.
11. If channels appear, repackage the tissue.

### Example 7

### Determining in Rabbit the In Vivo Release Rate of Triclosan from Anti-Infective-Treated Dermis

### Objective

The objective of this study is to evaluate the in-vivo release rate of the Triclosan from anti-infective-treated dermis when implanted subcutaneously in the abdomen in New Zealand White rabbits at 18 hours, 24 hours, and 48 hours.

### Method

Prior to surgery, 6 animals were weighed and ranked according to weight. Each animal was randomly assigned to one of the treatment groups. Randomization was per Table 6. Treatment groups are defined in Table 7.

TABLE 6
Animal rank and group assignments

| Rank | Group |
|---|---|
| 1 | A |
| 2 | C |
| 3 | E |
| 4 | B |
| 5 | D |
| 6 | F |

TABLE 7
Treatment groups.

| Group | No. | Treatment | Dose | Location | Necropsy |
|---|---|---|---|---|---|
| A | 2 | Triclosan Dermis | 1 piece | Abdomen | 18 hours ± 1 hour |
| B | 2 | Triclosan Dermis | 1 piece | Abdomen | 24 hours ± 1 hour |
| C | 2 | Triclosan Dermis | 1 piece | Abdomen | 48 hours ± 1 hour |

Fasting: Animals were fasted at least 2 hours prior to surgery.
Anesthesia: On Day 1, animals were weighed and anesthetized with an intravenous injection of a ketamine/xylazine cocktail (77 mg/mL ketamine, 23 mg/mL xylazine) at 0.1 mL/kg. Isoflurane was administered by inhalation (mask or intubation) during surgery as needed.
Surgical Procedure: The abdomen, from the xyphoid process to the groin, of each animal was shaved, prepared with betadine and alcohol scrubs, and draped using aseptic surgical techniques. A 10 cm midline abdominal incision (laparotomy) was made approximately 2 cm below the xyphoid process to the pubic symphysis. Using blunt dissection, the subcutaneous layer was dissected and bilateral skin flaps were raised. The skin was retracted laterally. A subcutaneous soft tissue pocket was created by bluntly dissecting the dermis and superficial fascia apart from the anterior abdominal wall. Triclosan-Dermis mesh samples, prepared as described in Example 6, 5 cm×6 cm with a thickness between 1.0 mm and 2.5 mm, were trimmed to fit. The Triclosan-Dermis mesh was placed, with the dermis side facing the abdominal wall (epidermis side facing ventral). Each corner of the implant was sutured to the abdominal wall, as an overlay, using Simple interrupted nonabsorbable PROLENE® suture. The subcutaneous tissues were closed using multi-layer suturing with nonabsorbable sutures. The midline fascia was closed with a running 2-0 PROLENE® suture. The skin was closed with subcuticular absorbable sutures, MONOCRYL® Suture. Sutures were followed with application of Nexa-Ban surgical glue over the incision. Animals were allowed to recover before being returned to caging. Two unimplanted Triclosan-Dermis samples were used as controls.
Analgesia: Animals were administered Buprenorphine at 0.03-0.05 mg/kg intramuscularly or subcutaneously upon recovery from anesthesia. Additional Buprenorphine was administered at the discretion of the BTC Staff Veterinarian and/or Study Director.
Mortality/Morbidity: Animals were monitored twice daily for mortality/morbidity. Any animal judged moribund by the Study Director were euthanized.
Body Weights: All animals were weighed at randomization, prior to surgery on Day 1, and prior to euthanasia.
Euthanasia: At 18±1 hours, 24±1 hours, and 48±1 hours following surgery, animals were administered an intravenous bolus of commercial euthanasia solution. Euthanasia was performed according to BCOP 01-11-21-02-026.
Necropsy: Following euthanasia, the abdominal wall was shaved and prepped for aseptic harvest. The skin was incised along the midline opened and the implant areas identified. Any gross evidence of graft shrinkage, inflammation, infection, fibrosis, seromas, nodules, or calcification were noted. The entire implant was carefully freed from the abdominal wall and removed. All of the explants were visually checked to record the gross observations on the condition of the implant and surrounding area at the time the implant was excised. Digital photographs of the implant ex vivo with ID label were taken. From each explant, an 8 mm diameter punch was used to harvest an 8 mm diameter biopsy sample. This sample was placed into a sterile mangar foil pouch and sealed. The location of the harvested specimens were marked on a drawing. The foil pouch was then placed into a sterile Tyvek pouch and sealed. This was the sample for the ZOI test. The Tyvek pouch was stored at −80° C. until analyzed. The remaining sample (3-5 g) was used for GC-MS analysis. This sample was placed into a pre-weighed sample vial and the mass recorded. These samples were stored at 80° C. until analyzed. Each explant specimen was identified by: the animal number, the duration of implantation, implant number, and designated testing. The numbering system was: Animal number-Time point-Implant-Test. These specimens were then analyzed.
Specimen preparation: The Triclosan (TCS) content of fourteen dermis specimen (12 implants, 2 controls) was determined. The dermis sample of approx 100-200 mg was put into centrifuge vial, covered with 10 ml of methanol and was fortified with 10 μg of ¹³C-TCS as an internal standard. The dermis was crushed with an Ultra-Turrax as effectively as possible. Another centrifuge vial with 10 ml of water was used to wash the Ultra-Turrax. The combined samples were further treated with ultrasonic radiation for 10 minutes. After filtration, the solution was diluted with methanol and analyzed by HPLC/MS/MS.
Quantification: Quantification was preformed by multi point calibration using ¹³C-TCS as internal standard.
Analytical standards: Calibration samples with a total volume of 1 ml were prepared with the following amounts by dilution of the TCS spike solution:

TABLE 9
Calibration standards.

| Standard | | | |
|---|---|---|---|
| concentration | ¹³C-TCS-spike-solution | ¹³C-TCS-ISTD-solution | Water |
| [ng/m] | [μl] | [μl] | [μl] |
| 1 | 5 | 10 | 985 |
| 5 | 25 | 10 | 965 |
| 10 | 50 | 10 | 940 |
| 20 | 100 | 10 | 890 |
| 50 | 250 | 10 | 740 |
| 100 | 500 | 10 | 490 |

Results: The determination of Triclosan (TCS) in 14 dermis samples was performed with HPLC/MS/MS after breakup and extraction with methanol under ultrasonic treatment. The measurement and quantification of TCS was possible in all sample extracts. Amounts of Triclosan were calculated in μg/g (=ppm). In addition, the density of the samples (in mg/cm²) was determined and the Triclosan amount is also given in μg/cm². Results are shown in Table 10.
Zone of inhibition (ZOI) assay: To determine if the implant specimen and control specimen display antimicrobial activity when investigated in an agar diffusion growth inhibition assay against the MRSA strain of Staphylococcus aureus. The method of the assay is described in Example 3. Results are shown in Table 10.

TABLE 10
Determined amounts of TCS in dermis samples,

| | | | | Abs. | | | |
|---|---|---|---|---|---|---|---|
| | Hours | Sample | Sample | Triclosan | Triclosan | Triclosan | |
| Sample | implanted | weight | density | amount | conc. | conc. | ZOI |
| name | in vivo | [mg] | [mg/cm²] | [μg] | [μg/g] | [μg/cm²] | [mm] |
| Implant #3 | 18 | 187.1 | 212 | 4.49 | 24.0 | 5.08 | 25 |
| Implant #4 | 18 | 169.6 | 162 | 8.54 | 50.4 | 8.18 | 26 |
| Implant #1 | 24 | 164.6 | 198 | 4.09 | 24.8 | 4.92 | 17 |
| Implant #2 | 24 | 173.7 | 173 | 3.22 | 18.5 | 3.21 | 19 |
| Implant #5 | 48 | 162.7 | 171 | 0.0397 | 0.244 | 0.04 | 14 |
| Implant #6 | 48 | 163.3 | 117 | 0.031 | 0.190 | 0.0222 | 14 |
| Control #1 | 0 | 161.9 | 191 | 26.4 | 163 | 31.16 | 28 |
| Control #2 | 0 | 181.7 | 195 | 30.5 | 168 | 32.67 | 28 |

Together results demonstrate that, over time, Triclosan may exude off of the implant and, hence, into the surrounding tissue.

While specific embodiments of the subject invention have been discussed, the above specification is illustrative and not restrictive. One skilled in the art will appreciate that numerous changes and modifications can be made to the invention, and that such changes and modifications can be made without departing from the spirit and scope of the invention. The full scope of the invention should be determined by reference to the claims, along with their full scope of equivalents, and the specification, along with such variations.

Each patent, patent application, and publication cited or described in the present application is hereby incorporated by reference in its entirety as if each individual patent, patent application, or publication was specifically and individually indicated to be incorporated by reference.

1. A biologic matrix comprising a slowed release anti-infective agent.

2. The biologic matrix of claim 1, wherein the biologic matrix is an acellular biologic matrix.

3. The acellular biologic matrix of claim 2, wherein the acellular biologic matrix is derived from tissue selected from the group consisting of: dermal, fascia, dura, pericardia, tendons, ligaments, and muscle.

4. The acellular biologic matrix of claim 3, wherein the acellular biologic matrix is an acellular dermal matrix.

5. The acellular biologic matrix of claim 2, wherein the acellular biologic matrix is hydrated with a solution of specially denatured alcohol and water in a ratio of alcohol to water selected from the group consisting of: 20:80; 25:75; 30:70; 35:65; 40:60; 45:55; 50:50; 55:45; 60:40; 65:35; 70:30; 75:25; 80:20; 85:15; 90:10; and 95:05.

6. The acellular biologic matrix of claim 5, wherein the acellular biologic matrix is hydrated with a 70% specially denatured alcohol and 30% water solution.

7. (canceled)

8. The acellular biologic matrix of claim 5, wherein the acellular biologic matrix is hydrated with a 40% specially denatured alcohol and 60% water solution.

9.-18. (canceled)

19. The acellular biologic matrix of claim 6, wherein the hydrated biologic matrix comprising anti-infective is vacuum-dried from about one minute to about six minutes.

20. The acellular biologic matrix of claim 6, wherein the hydrated biologic matrix comprising anti-infective demonstrates anti-microbial activity in a zone-of-inhibition assay against gram positive bacteria.

21. The acellular biologic matrix of claim 20, wherein the zone-of-inhibition assay comprises an 8 mm diameter disc of the dermal material applied to an agar plate treated with 0.5 mL of a 1:1000 dilution of Staphylococcus aureus and the zone-of-inhibition of the gram positive bacteria is at least 10 mm within about 48 hours.

22. The acellular biologic matrix of claim 6, wherein the treated acellular biologic matrix demonstrates toxicity against gram positive bacteria, wherein a log reduction of greater than 1.0 is observed within 24 hours.

23. The acellular biologic matrix of claim 22, wherein a log reduction greater than 2.0 is observed within 24 hours.

24. A method of treating an acellular biologic matrix with an anti-infective agent, comprising:
(a) hydrating an acellular biologic matrix with a solution of about 30-70% specially denatured alcohol; and
(b) dipping the acellular biologic matrix in a solution comprising an anti-infective agent.

25. A method of treating an acellular biologic matrix with an anti-infective agent, comprising:
(a) hydrating an acellular biologic matrix with a solution of about 30-70% specially denatured alcohol; and
(b) soaking the acellular biologic matrix in a solution comprising an anti-infective agent for at least about five seconds and less than 1 hour.

26.-29. (canceled)

30. The acellular biologic matrix of claim 2, wherein the acellular biologic matrix is suitable for implantation into a human patient.

31. The acellular biologic matrix of claim 30, wherein the acellular biologic matrix is suitable for use in a surgical procedure selected from the group consisting of: hernia repair, abdominal wall repair; breast reconstruction, cranial reconstruction, maxillary reconstruction, facial reconstruction, urologic reconstruction, gynecologic reconstruction, pulmonary reconstruction, bladder neck suspension, tendon repair, chronic wound care, acute wound care, burn care, dura repair and replacement, gastrointestinal reconstruction; parastomal reinforcement and repair, trauma repair, diabetic ulcer and chronic venous insufficiency ulcer.

32. The acellular biologic matrix of claim 31, wherein the hernia is selected from the group consisting of: ventral, inguinal, paraesophageal, incisional, and hiatal

33. The acellular biologic matrix of claim 31, wherein the anti-infective agent is slowly released from the acellular biologic matrix after implantation into the human patient.

34. The acellular biologic matrix of claim 30, further comprising at least one additional anti-infective agent.

35. The acellular biologic matrix of claim 34, wherein the at least one additional anti-infective agent is chlorhexidine gluconate.

36.-37. (canceled)

38. The biologic matrix of claim 2, wherein the anti-infective agent is encapsulated in a selectively degradable polymer affixed to the acellular biologic matrix.

39. The acellular biologic matrix of claim 38, wherein the selectively degradable polymer is selected from the group consisting of: poly lactic glycolic acid copolymer, polycaprolactone, alginate, gelatin, collagen, carboxymethylcellulose, and hyaluronic acid.

40. The method of claim 25, further comprising lyophilizing the biologic matrix treated with an anti-infective agent.

41. The acellular biologic matrix of claim 3, wherein the tissue is derived from a mammalian source selected from the group consisting of: humans, non-human primates, pigs, cows, horses, goats, sheep, dogs, cats, rabbits, guinea pigs, gerbils, hamsters, rats, or mice.

42.-46. (canceled)

47. The acellular biologic matrix of claim 2, wherein the acellular biologic matrix has been disinfected.

48. The acellular biologic matrix of claim 2, wherein the acellular biologic matrix has been sterilized.

49. (canceled)
